(19) United States
(12) Patent Application Publication
(10) Pub. No.: US 2026/0315497 A1
(43) Pub. Date: Oct. 15, 2026

(54) COMPOSITION FOR TREATING EPILEPSY

(71) Applicant: CanaQuest Medical Corp., Mississauga, Ontario (CA)

(72) Inventor: Richard Rusiniak, Mississauga, Ontario (CA)

(73) Assignee: CanaQuest Medical Corp., Mississauga, Ontario (CA)

(21) Appl. No.: 19/119,087

(22) PCT Filed: Oct. 4, 2023
(86) PCT No.: PCT/CA2023/051315

§ 371 Date: Apr. 7, 2025

Related U.S. Application Data
(60) Provisional application No. 63/414,421, filed on Oct. 7, 2022.
(60) Provisional application No. 63/428,157, filed on Nov. 28, 2022.

(51) Int. Cl. A61K 31/00; A61K 9/107; A61K 31/202; A61P 25/08

(52) U.S. Cl. CPC A61K 31/658; A61K 9/107; A61K 31/202; A61P 25/08

(57) ABSTRACT

A composition for use in the treatment of epilepsy, the composition comprising at least one cannabinoid and at least one omega-3 fatty acid.

### BACKGROUND

Epilepsy deleteriously affects millions of people worldwide.

Many epileptics have seizures that are not readily controlled with conventional medications.

In animal models and clinical trials, cannabinoids, and especially cannabidiol (CBD) have been shown to stop seizures.

### SUMMARY OF THE INVENTION

Forming one aspect of the invention is a composition for use in the treatment of epilepsy, the composition comprising at least one cannabinoid and at least one omega-3 fatty acid.

According to another aspect, the composition can be in the form of an oil in water emulsion.

According to another aspect, the at least one cannabinoid is dissolved in the at least one omega-3 fatty acid and the at least one omega-3 fatty acid can define the oil in the oil in water emulsion.

According to another aspect, the at least one cannabinoid can comprise cannabidiol (CBD).

According to another aspect, the at least one omega-3 fatty acid can comprise one or more of Docosahexaenoic acid (DHA) and Eicosapentaenoic acid (EPA).

According to another aspect, the weight ratio of the at least one cannabinoid to the at least one omega-3 fatty acid can be about 1:1

According to another aspect, the weight ratio of DHA to EPA can be about 1:1

Advantages, features and characteristics of the present invention will become apparent upon a review of the following detailed description, with reference to the figures, the latter being briefly described hereinafter.

### BRIEF DESCRIPTION OF THE FIGURES

FIG. 1 shows the effects of two preparations of CBD in the MES model in mice.

### DETAILED DESCRIPTION

A composition was prepared with the following ingredients, expressed in weight %

| | |
|---|---|
| Fish Oil (DHA & EPA) | 3.5% |
| CBD - | 2.0% |
| Stabilizer - | 6.5% |
| Distilled water - | 88.0% |
| Total | 100.0% |

The CBD was isolate powder. The fish oil was unoxidized (fresh) with roughly equal amounts DHA and EPA. The stabilizer was LSO NanoStabilizer obtained from Sonomechanics (USA).

The above created an oil in water emulsion as follows:

$$particle size 66.2 + / - 0.1 nm$$

$$polydispersity index = 0.107 + / - 0.008$$

### EXPERIMENTAL

The composition was tested using the Maximal Electroshock Seizure (MES) Model. This is an established procedure to provide an indication of a compound's ability to prevent seizure spread.

### Initial Tests

Subjects Male, CF-1 male mice (18-25 grams) were used as subjects. They were housed under standard conditions in the vivarium at the CCBR. They were given 2 days (minimum) to acclimate before testing began.

Drugs and Doses CBD was provided in pure form and in the described composition. Two four-point dose-response curves were done, one for the pure form CBD and one for the described composition. Ten mice were tested at each dose. (Mice were not re-used.) Compounds were injected intraperitoneally. Testing was done 2 hours after injection. Drugs were injected at a constant volume, and were diluted as necessary using a vehicle of 1:1:18 (Cremophor:alcohol:saline). Dilution is necessary to achieve volumes large enough for injection. Doses were: 0 (vehicle), 25, 50, 100, 200 mg/kg. A 400 mg/kg dose was added later to extend the standard CBD curve.

MES Procedure The MES stimulus consisted of 0.2 sec of 60 Hz since-wave current administered at an intensity of 50 mA via corneal electrodes. The percentage of mice protected against the tonic hindlimb extension component of the resulting seizure was measured. Mice were then euthanized following the procedure.

Toxicity Assessment Ataxia (impaired movement) was assessed using the Loscher scale and was done just before the MES test.

### Results

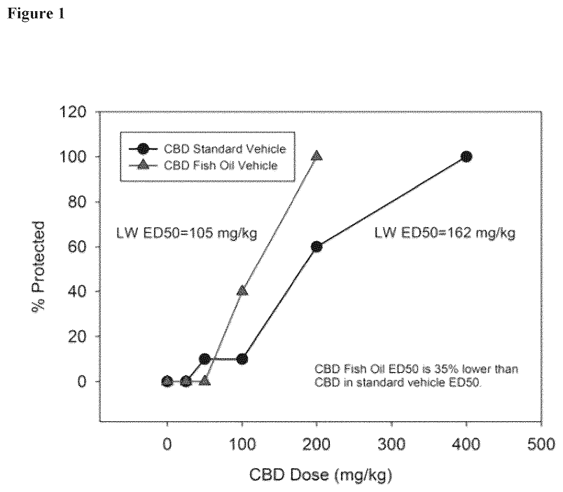

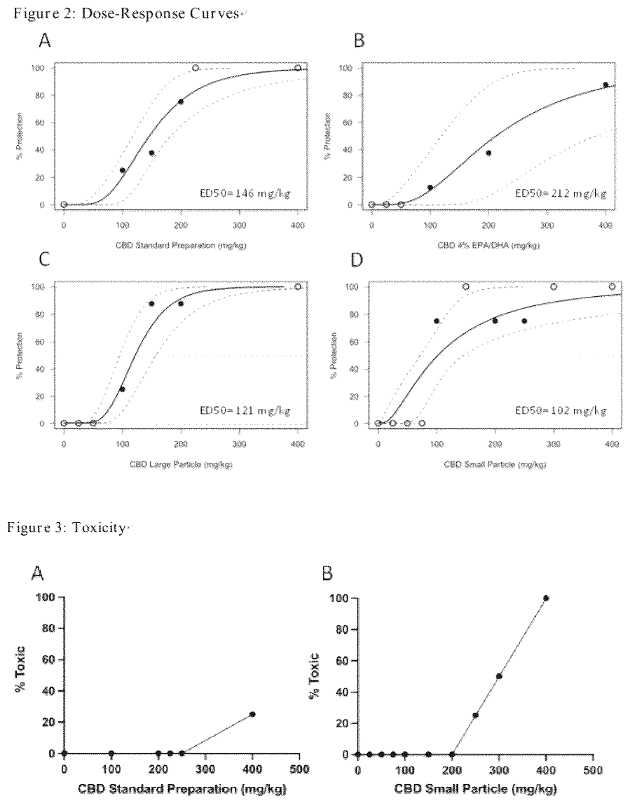

Antiseizure effects FIG. 1 shows dose-response curve for the antiseizure effects of the two forms of CBD in the MES test in mice. The ED50 (effective dose to protect 50% of mice) for regular CBD was 162 mg/kg and the ED50 for the described composition was 105 mg/kg. The ED50 for the described formulation was considerably lower. This difference was statistically significant.

Toxicity No mice showed any signs of toxicity in the standard CBD group at any dose. In the new composition group two subjects showed moderate signs of toxicity (Loscher scale 3) at the highest dose.

Discussion The described composition appeared more effective than regular CBD.

The described composition produced some toxicity at the highest dose, which suggests that more CBD was getting into the brain.

### Further Tests

Subjects Male, CF-1 male mice (18-25 grams) were used as subjects. They were housed under standard conditions in a vivarium at the University of Toronto (CCBR). They were given 2 days (minimum) to acclimate before testing began.

Drugs and Doses CBD was provided in the form of: 1) standard CBD isolate, 2) CBD in DHA/EPA without emulsification (2% DHA, 2% EPA, 12% “stabilizer”), 3) CBD emulsified in DHA/EPA to produce larger particles (66.2 nm diameter) and 4) CBD emulsified in DHA/EPA to produce smaller particles (32.7 nm diameter). Drugs were injected intraperitoneally at a constant volume, and were diluted as necessary using a vehicle of 1:1:18 (Cremophor:alcohol:saline). Dilution is necessary to achieve volumes large enough for injection. Doses of CBD ranged from 0 (vehicle) to 400 mg/kg. As in our past studies, the MES stimulus was given 2 hours after injection. Dose-response curves were done for each formulation.

MES Procedure The MES stimulus consisted of 0.2 sec of 60 Hz since-wave current administered at an intensity of 50 mA via corneal electrodes. The percentage of mice protected against the tonic hindlimb extension component of the resulting seizure was measured. Mice were used only once and euthanized following the procedure.

Toxicity Assessment Ataxia (impaired movement) was assessed using the Loscher scale and was done just before the MES test. Loscher scores of 2-5 were scored a “toxicity present”. Statistical Analysis ED50 for protection (i.e., 50% of mice show protection) was determined using the Litchfield and Wilcoxon method as implemented by the LW1949 package (ver. 1.1.0) in the RStudio (2022.07.2) programming environment (Adams et al., 2016; Litchfield and Wilcoxon, 1949). ED50's are presented with 95% confidence intervals. The ED50 for the different formulations were compared using the z-test method described by Austin and Hux (2002). A ztest statistic greater than 1.96 (P<0.05) was considered significant when 95% confidence intervals overlapped.

The Litchfield/Wilcoxon method could not be applied to the toxicity data for technical reasons, so TD50s were calculated from line plots.

### Results

Antiseizure effects FIGS. 2A, B, C and D shows dose-response curves for the four formulations in the MES test in mice.

The ED50 (effective dose to protect 50% of mice) for regular CBD in the standard vehicle was 146 mg/kg (FIG. 2A).

The ED50 for CBD in DHA/EPA was tested to determine whether DHA/EPA alone increased CBD's effectiveness. The ED50 for CBD in DHA/EPA without emulsification was 212 mg/kg, which is higher than the ED50 for CBD alone (FIG. 1B). This difference when compared to CBD in the standard vehicle was not significant, but the findings do not suggest that adding DHA/EPA improves CBD's anti-seizure effects.

The ED50 for the large particle emulsification in DHA/EPA was 121 mg/kg, which is lower than the ED50 for CBD alone (FIG. 2C). This difference when compared to CBD in the standard vehicle was not significant.

The ED50 for the small particle emulsification in DHA/EPA was 102 mg/kg (FIG. 2D). This difference when compared to CBD in the standard vehicle was significant.

Taken together, the results of the last two experiments suggest that emulsification increases the effectiveness of CBD, and that emulsification to a smaller particle size is better than emulsification to a larger particle size

Toxic Effects FIGS. 3A and B present the toxicity data for CBD alone and CBD emulsified to smaller particles. Neither of the other preparations showed any toxicity up to doses of 400 mg/kg.

As indicated, CBD alone showed a TD25 at 400 mg/kg, the highest dose tested. It is speculated that the TD50 would be higher than 400 mg/kg (FIG. 3A).

The TD50 for CBD emulsified to small particles was 300 mg/kg (FIG. 3B). This is lower than the TD50 for CBD alone, consistent with the idea that more CBD crosses barriers in the small particle formulation. The therapeutic index (TD50/ED50) for CBD in the small particle is still about 3, which is in the acceptable range.

### DISCUSSION

CBD emulsified into small particles was the most effective formulation.

The ED50 for CBD emulsified into small particles was the lowest, and was significantly different from CBD alone.

Adding DHA/EPA to CBD without emulsification did not lower the ED50 for CBD.

The results suggest that micro-emulsification increases the potency of CBD, but that adding DHA/EPA to CBD without emulsification does not.

### Variations

Whereas a specific composition is shown, variations are possible.

Without limitation in this regard, other sources of omega-3 fatty acid can be utilized, other stabilizers can be utilized and the weight ratio of fatty acid to cannabinoid can be varied. The ratio of cannabinoid to omega 3 can vary between 1.5:1 and 4:1.

Further, whereas intraperitoneal injection was utilized, other forms are injection could be utilized. For example, it is well known that many drugs introduced intraperitonally can also be introduced orally.

Accordingly, the invention should be understood to be limited only by the accompanying claims, purposively construed.

1. A composition for use in the treatment of epilepsy, the composition comprising at least one cannabinoid and at least one omega-3 fatty acid.

2. The composition of claim 1, further comprising water and in the form of an oil in water emulsion.

3. The composition of claim 2, wherein the at least one cannabinoid is dissolved in the at least one omega-3 fatty acid and the at least one omega-3 fatty acid defines the oil in the oil in water emulsion.

4. The composition of claim 3, wherein the at least one cannabinoid comprises cannabidiol (CBD).

5. The composition of claim 4, wherein the at least one omega-3 fatty acid comprises one or more of Docosahexaenoic acid (DHA) and Eicosapentaenoic acid (EPA).

6. The composition of claim 4, wherein the weight ratio of the at least one cannabinoid to the at least one omega-3 fatty is acid is about 1:1

7. The composition of claim 5, wherein the weight ratio of DHA to EPA is about 1:1
